(19) United States
(12) Patent Application Publication
(10) Pub. No.: US 2010/0036267 A1
(43) Pub. Date: Feb. 11, 2010

(54) ANALYSIS DEVICE AND ANALYZING METHOD FOR AUTONOMIC NERVE STATE

(76) Inventors: Wen-Chien Liao, Taipei (TW); Yu-Chieh Kao, Taipei (TW); Fu-Shan Jaw, Taipei (TW); Chii-Wann Lin, Taipei (TW)

Correspondence Address: Muncy, Geissler, Olds & Lowe, PLLC, P.O. BOX 1364, FAIRFAX, VA, 22038-1364, US

(21) Appl. No.: 12/536,898

(22) Filed: Aug. 6, 2009

(30) Foreign Application Priority Data
Aug. 7, 2008 (TW) 097130157

(51) Int. Cl. A61B 5/02

(52) U.S. Cl. 600/500

(57) ABSTRACT

A detecting device for detecting an autonomic nervous state of an examinee includes a measuring unit, an analog-to-digital converting (ADC) unit and a digital processing unit. The measuring unit measures a vital sign of the examinee to output a vital sign signal. The ADC unit is coupled to the measuring unit for receiving and converting the vital sign signal from analog to digital to output a digitalized vital sign signal. The digital processing unit is coupled to the ADC unit for receiving and processing the digitalized vital sign signal to obtain a detecting result. The detecting result represents the autonomic nervous state and organic states of the examinee.

### CROSS REFERENCE TO RELATED APPLICATIONS

This Non-provisional application claims priority under 35 U.S.C. §119(a) on Patent Application No(s). 097130157 filed in Taiwan, Republic of China on Aug. 7, 2008, the entire contents of which are hereby incorporated by reference.

### BACKGROUND OF THE INVENTION

1. Field of Invention

The present invention relates to a detecting device, an analysis device, and an analyzing method for detecting a nervous state and, in particular, to a detecting device, an analysis device, and an analyzing method of an autonomic nervous state.

2. Related Art

Since the lifestyle today has transformed from agriculture and fishery into industry and commerce in urban areas, it has become easier for people to develop the cardiovascular diseases because of unhealthy diet and lack of exercise. Besides, for the advance in biotechnology and the increase in life expectancy, the aging population has also been increasing, and it is easier for the elders to develop the cardiovascular diseases.

There are various kinds of equipment for examining various diseases. The common devices for examining the cardiovascular functions are, for example, the electrocardiograph used to examine the heart and the oximeter used to examine the blood vessel.

The electrocardiograph can examine the cardiac rhythm as the heart is having periodic contraction, and the cardiac rhythm can be presented in the waveform, which is called the electrocardiogram. The electrocardiogram may be used to determine whether the cardiac function is at a normal state.

The oximeter is used to examine the oxygen concentration in blood (SPO2). The measured SPO2 can be used to determine the states of the blood circulation function and the oxygen supply in blood, and to determine whether the oxygen transmitted to the brain tissue is sufficient.

However, the examining results obtained from both the electrocardiograph and the oximeter are needed to be analyzed by the professionals. If the inspection results from the instruments can be judged by the professionals, the mistakes made by people can be reduced. Furthermore, if a further vital sign is found in the examining system consisted of the electrocardiograph and the oximeter, it can be used to find the symptoms of the diseases at an early stage.

### SUMMARY OF THE INVENTION

In view of the foregoing, the present invention is to provide a detecting device, an analysis device, and an analyzing method that examine an autonomic nervous state.

To achieve the above, a detecting device for detecting an autonomic nervous state of the present invention is used on an examinee. The detecting device includes a measuring unit, an analog-to-digital converting (ADC) unit, and a digital processing unit. The measuring unit measures the pulse of the examinee to output a vital sign signal. The ADC unit is coupled to the measuring unit for receiving the vital sign signal and converting the vital sign signal from analog to digital to output a digitalized vital sign signal. The digital processing unit is coupled to the ADC unit for receiving the digitalized vital sign signal and processing the digitalized vital sign signal to obtain a detecting result. The detecting result represents the autonomic nervous state and organic states of the examinee.

In addition, the present invention also discloses an analysis device for analyzing an autonomic nervous state, which is used with a measuring device that measures a vital sign of an examinee to output a vital sign signal. The analysis device of a vital sign includes an ADC unit and a digital processing unit. The ADC unit receives a vital sign signal from the measuring device, and converts the vital sign signal from analog to digital to output a digitalized vital sign signal. The digital processing unit is coupled to the ADC unit for receiving the digitalized vital sign signal and processing the digitalized vital sign signal to obtain a detecting result. The detecting result represents the autonomic nervous state and organic states of the examinee.

To achieve the above, the present invention further discloses an analyzing method of an autonomic nervous state. The analyzing method includes the steps of receiving a vital sign signal from a measuring device, which measures a vital sign of an examinee to output the vital sign signal; converting the vital sign signal from analog to digital to output a digitalized vital sign signal; and processing the digitalized vital sign signal to obtain a detecting result, which represents the autonomic nervous state and organic states of the examinee.

In a preferred embodiment, the detecting result is generated by the processes such as the short-time Fourier transform, the Hilbert-Huang transform, or an entropy analysis. These signal processing methods can process a non-periodic input signal, and specifically the Hilbert-Huang transform and the entropy analysis can process a non-steady state input signal. Thus, the vital sign detecting signal may be analyzed and used to track the physiological conditions of the examinee in real-time. For example, such real-time analyzing pattern may be used to track the chronic patients or the high risk population, or used for the application of in-home care. It may also be used on an ambulance or a fire fighting truck to initially determine the conditions of the patient.

Moreover, a detecting device for detecting an autonomic nervous state of the present invention is used on an examinee. The detecting device includes a body and an infrared measurement unit. The body has a concave and the infrared measurement unit is disposed in the concave. When the examinee's finger is placed in the concave and making contact with the infrared measurement unit, the infrared measurement unit measures the pulse of the examinee to output a vital sign signal.

As described above, the detecting device, analysis device, and analyzing method of an autonomic nervous state of the present invention are to process and analyze the detected vital signs by a frequency domain analysis or an entropy analysis for example, so that the signs of the autonomic nervous state can further be obtained. The organic or systematic states in the human body may be determined by a spectrogram or the entropy analyzing result.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a block diagram illustrating a detecting device according to a preferred embodiment of the present invention;

FIG. 2 is a functional block diagram illustrating function processing blocks of the digital processing unit in FIG. 1;

FIGS. 3 and 4 are schematic views illustrating a spectrogram;

FIG. 5 is a schematic view illustrating an entropy analysis result;

FIG. 6 is a block diagram illustrating an analysis device according to the preferred embodiment of the present invention; and

FIGS. 7 and 8 are schematic views illustrating the detecting devices according to the preferred embodiment of the present invention.

### DETAILED DESCRIPTION OF THE INVENTION

The present invention will be apparent from the following detailed description, which proceeds with reference to the accompanying drawings, wherein the same references relate to the same elements.

The autonomic nerve of human beings is in charge of each organ and the respiratory system, circulatory system, digestive system, endocrine system, external secretion system, and excretory system. There are two kinds of autonomic nerve: one is sympathetic nerve and the other is parasympathetic nerve. When the autonomic nerve starts to adjust and control by itself, the sympathetic nerve and the parasympathetic nerve may not be in a balanced state. Hence the sympathetic nerve and the parasympathetic nerve of the autonomic nerve are analyzed to acquire the actual condition of each organ or system of the examinee. The analysis is performed by pre-estimating the time-frequency variation of each organ or system of the examinee or by calculating the consumed entropy. Therefore, the measured autonomic nervous electric signal may be used to further understand the condition of each organ or system at the preliminary stage of the disease.

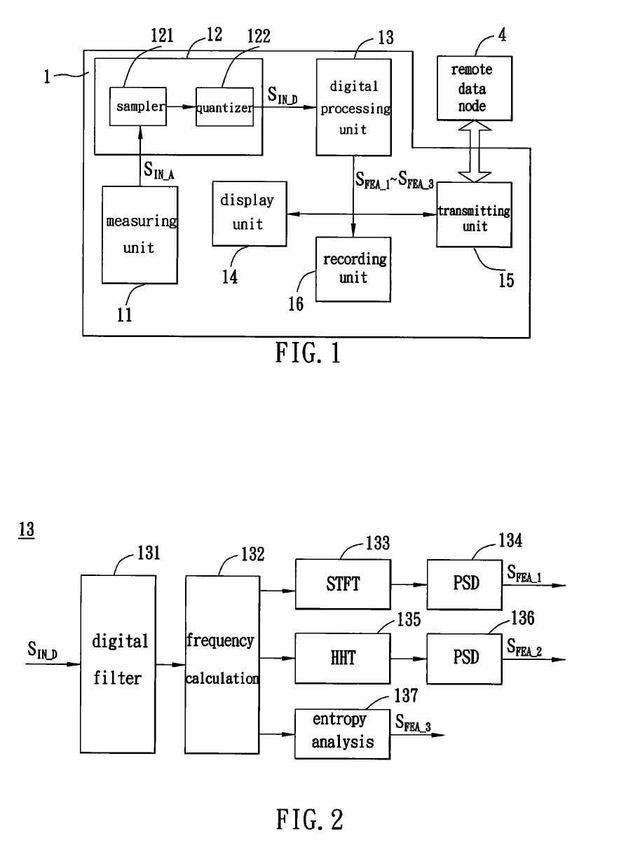

As shown in FIG. 1, a detecting device 1 of an autonomic nervous state includes a measuring unit 11, an analog-to-digital converting (ADC) unit 12, a digital processing unit 13, a display unit 14, a transmitting unit 15, and a recording unit 16.

The measuring unit 11 measures a vital sign of an examinee to output a vital sign signal SIN_A. The ADC unit 12 is coupled to the measuring unit 11 for receiving the vital sign signal SIN_A, and converting the vital sign signal SIN_A from analog to digital to output a digitalized vital sign signal SIN_D. The digital processing unit 13 is coupled to the ADC unit 12 for receiving the digitalized vital sign signal SIN_D, and processing the digitalized vital sign signal SIN_D to obtain a detecting result, which represents the autonomic nervous state and the organic states of the examinee.

The vital signs measured by the measuring unit 11 may be the pulse, heartbeat, oxygen concentration in blood (SPO2), white blood cell concentration, carbon dioxide concentration in blood, respiration rate, brainwave, and radiation (infrared) of each organ. The common measuring devices for measuring the cardiovascular system are the electrocardiograph and the oximeter. Naturally, the other measuring devices for measuring the cardiovascular system, other organs, or other systems may be used as the measuring unit 11. For example, an infrared autonomic nerve scanner or an extra-cranium electrode brainwave measuring device that can measure the vital signs of the autonomic nervous state can be used.

For easily tracking the examinee, the measuring unit 11 is preferably non-invasive. In addition, since the examining procedure of the professional examining device is complicated, a portable or small-sized measuring device is easier to use. For example, the oximeter is able to measure the blood oxygen signal by measuring the finger of the examinee. Furthermore, the infrared autonomic nerve scanner selects correct measuring result by scanning the arterial pulse of the fingers through infrared, diversity detecting, and comparing the measuring value of the two fingers.

In addition, the ADC unit 12 includes a sampler 121 and a quantizer 122 in the embodiment. The sampled vital sign signal SIN_A is quantized by the quantizer 122, so that the quantizer 122 may output the digitalized vital sign signal SIN_D.

The digital processing unit 13 is coupled with the quantizer 122 of the ADC unit 12 for receiving the digitalized vital sign signal SIN_D. The digital processing unit 13 may perform the frequency spectrum analysis and/or the entropy analysis on the digitalized vital sign signal SIN_D. Before analyzing, the digital processing unit 13 may use the digital filter 131 on the signal.

For the frequency spectrum analysis, the digital processing unit 13 performs the frequency calculation 132 and the frequency domain transform on the digitalized vital sign signal SIN_D to obtain a vital sign time-frequency spectrum. After that, the power spectrum density (PSD) of the vital sign time-frequency spectrum is calculated for obtaining a spectrogram with the autonomic nervous state. While the PSD is used to enhance the vital sign time-frequency spectrum, other ways to enhance the vital sign time-frequency spectrum may also be used. If the vital sign time-frequency spectrum is clear enough, it is acceptable not to enhance the signal.

The vital sign signal SIN_A and the digitalized vital sign signal SIN_D are not completely regular periodic signals. If the inputted digitalized vital sign signal SIN_D is stationary, the digital processing unit 13 may perform the short time Fourier transform (STFT) 133 on the digitalized vital sign signal SIN_D to obtain the time-frequency graph, and then the PSD 134 of the time-frequency graph is calculated for obtaining a spectrogram SFEA_1 with the autonomic nervous state.

If the inputted signal changes non-steadily or irregularly, it will lose the time domain information by directly performing Fourier transform. The STFT is to divide the inputted signal into small segments and to carry out the Fourier transform by segment to obtain the time domain information. The STFT mainly uses a horizontal shift parameter of the time axis to cover the whole time axis so as to obtain the regional information of the signal.

Additionally, if the inputted digitalized vital sign signal SIN_D is a non-stationary signal or a plurality of analyzing results desired to be obtained by further analyzing the inputted digitalized vital sign signal SIN_D, the digital processing unit 13 may carry out a Hilbert-Huang transform (HHT) 135 on the digitalized vital sign signal SIN_D to obtain the vital sign time-frequency spectrum. The PSD 136 of the vital sign time-frequency spectrum is then calculated for acquiring a spectrogram SFEA_2 with the autonomic nervous state.

The HHT is different than the STFT. The HHT is suitable for the analysis of a non-stationary signal or a non-linear signal. The HHT breaks the inputted signal into several IMFs and each IMF represents the agitation of the signal in different scales and actually reflects the original properties of the signal.

On the other hand, as for the entropy analysis, the digital processing unit 13 performs the frequency calculation 132 and the entropy analysis 137 on the digitalized vital sign signal SIN_D to obtain the analyzing result SFEA_3 with the autonomic nervous state. In the embodiment, after the digital processing unit 13 carries out the entropy analysis on the digitalized vital sign signal SIN_D, a mean square error calculation is performed to obtain the entropy analyzing result SFEA_3.

Entropy may be used as the scale of “regularity.” If the input signal is a continuous sequence with simpler regularity, the entropy will be closer to zero; on the contrary, if the entropy is more complex, its value will be higher.

The STFT, the HHT, or the entropy analysis in the above embodiment can both process the non-periodic inputted signals, and particularly the HHT and the entropy analysis can further process the non-stationary inputted signal. Thus, the detecting device 1 may analyze and measure the vital sign detecting signal, and track down the physiological condition of the examinee in real-time. For example, the detecting device 1 may be used to track the chronic patients or the high risk population, or used to determine the initial condition when in an emergency situation.

A display unit 14 is coupled with the digital processing unit 13 for displaying the spectrogram and/or the entropy analyzing results SFEA_1 to SFEA_3, so that the user, the examinee, and/or the doctor may observe the examining result. Additionally, a transmitting unit 15 is coupled with the digital processing unit 13 for transmitting the spectrogram and/or the entropy analyzing results SFEA_1 to SFEA_3.

For example, the transmitting unit 15 and a remote data node 4 are both connected to the Internet so that the transmitting unit 15 may transmit data to the remote data node 4 through the Internet.

The recording unit 16 may be a non-volatile memory such as a hard disk, a magnetic disk, a compact disk, or a flash memory. The recording unit 16 may store a plurality of reference results, for example, the historic examining results of the examinee or other examinees. These historic data include the spectrogram and/or the entropy analyzing result that can help determine the condition of the examinee at the time the exam was taken. The digital processing unit 13 is coupled with the recording unit 16 for reading the reference results and comparing the detecting result and the reference result to determine the autonomic nervous state. If the determined state is severely abnormal, the digital processing unit 13 can control the transmitting unit 15 depending on the abnormality degree, and then the transmitting unit 15 emergently transmits the examining result to the remote data node 4. The remote data node 4 carries out an emergent processing after receiving the result. Furthermore, the remote data node 4 may record the historic examining result in the way similar to that of the recording unit 16 and provides the related information for the detecting device 1.

The digital processing unit 13 may perform the tendency prediction according to the time-frequency analyzing result and/or the entropy analyzing result. The tendency prediction result may be used to remind the user or used as a basis for safety check. The way of tendency prediction may predict the examining result of the examinee after a period of time in the future according to the related historic data, for example, predicting the frequency spectrum intensity or the entropy intensity of an organ at the corresponding frequency after a period of time, and further comparing the predicted result and the historic data to determine the state of the examinee after a period of time.

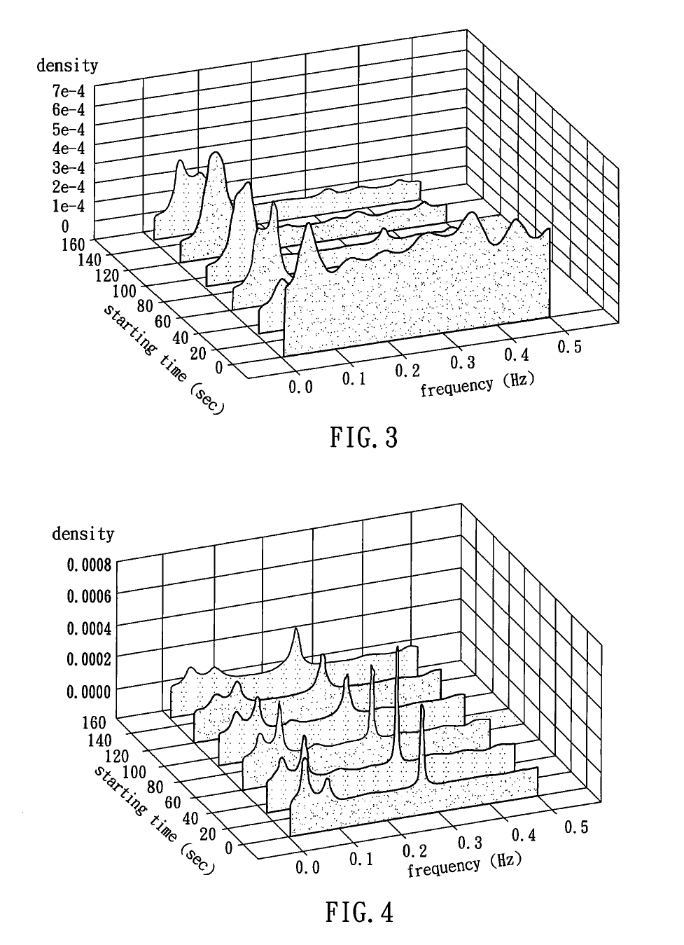

As shown in FIGS. 3 and 4, the spectrogram is used to examine the rhythm variation of heart rate for example. The spectrogram is the time-frequency power spectrum of the rhythm variation of heart rate, and the sympathetic nervous state and the parasympathetic nervous state of the autonomic nerve are corresponding to a very low frequency and a low frequency whenever, respectively. The examinee may determine whether his autonomic nervous system is at balance from the frequency band distribution of the time-frequency power spectrum according to the theory that the low frequency band (from 0.15 to 0.4 Hz) represents the parasympathetic nervous activity and the very low frequency band (from 0.01 to 0.04 Hz) represents the sympathetic nervous activity.

FIG. 3 is a spectrogram of a normal healthy person. FIG. 4 is a spectrogram of a patient with incontinence who already urinated. It is apparent that the corresponding intensities of the sympathetic nerves in these two figures are different. Similarly, the corresponding intensities of the parasympathetic nerves in these two figures are different.

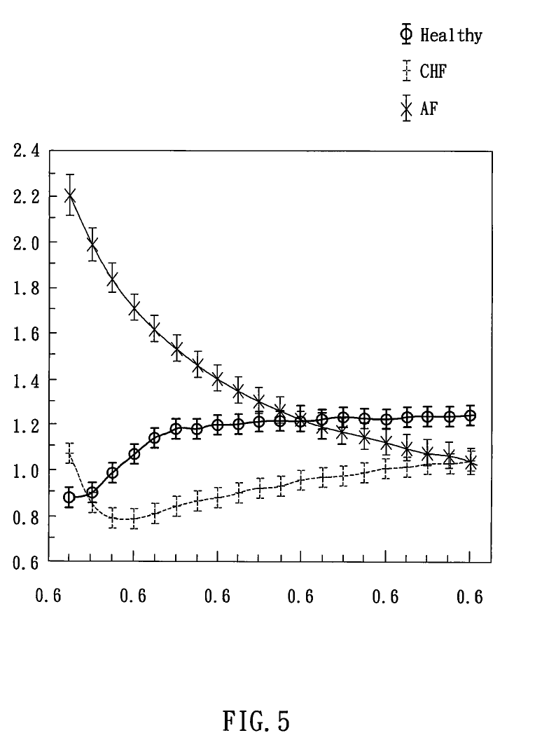

As shown in FIG. 5, the corresponding entropy of the patients with congestive heart failure (CHF) is different from that of the healthy examinees. Furthermore, the corresponding entropy of the patients with atrial fibrillation (AF) is different from that of the healthy examinees.

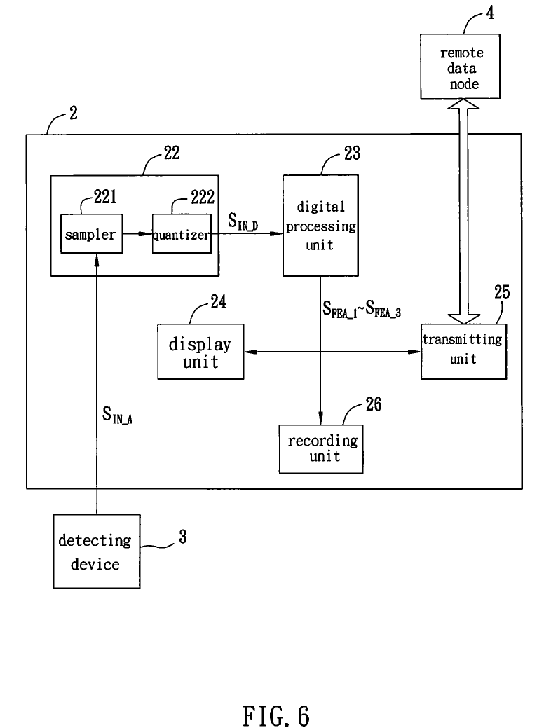

As shown in FIG. 6, an analysis device 2 of the autonomic nervous state is used with a detecting device 3. The measuring unit 11 itself of the above embodiment may be the detecting device 3. The analysis device 2 includes an ADC unit 22, a digital processing unit 23, a display unit 24, a transmitting unit 25, and a recording unit 26.

For example, these units are integrated or operated in the same device. The analysis device 2 may be a computer; the ADC unit 22 may be an interface adapting card; the digital processing unit 23 may be a subsystem operating the arithmetic in the computer (e.g. a CPU, a system-on chip, a memory, a motherboard, or a bus). The display unit 24 may be a subsystem processing the graphics (e.g. a graphic processor or a display); the transmitting unit 25 may be a subsystem for communication (e.g. a wired network module, a wireless network module, a MODEM module, or a peripheral bus). The recording unit 26 may be a hard disk drive, a disk, a disk drive, or a non-volatile memory. Additionally, the detecting device 2 may be a small electronic device, for example, the ADC unit 22 may be a single-chip converter and the digital processing unit 23 may be a digital processor, an ASIC, or a logic circuit. The display unit 24 is the graphic processor and a display panel. Moreover, the ADC unit 22 may be integrated with the digital processing unit 23 to be a digital processor with analog-to-digital conversion function.

Since the above elements have the same functions, effectiveness, and various aspects as those disclosed in FIG. 1, the detailed descriptions thereof will be omitted.

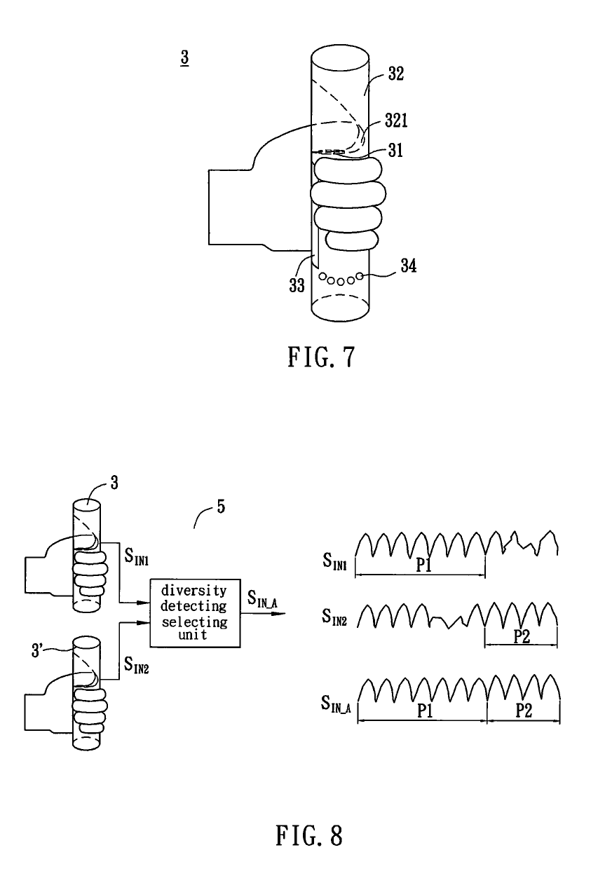

As shown in FIG. 7, the detecting device 3 includes a measuring unit 31, a body 32, a heating unit 33, and a display unit 34. The body 32 has a concave 321, and the measuring unit 31 is disposed in the concave 321. As the finger of the examinee enters into the concave 321 and makes contact with the measuring unit 31, the measuring unit 31 measures the vital signs of the examinee to output the vital sign signals.

For example, the measuring unit 31 may measure the pulse of the examinee by the infrared measurement device, which is highly sensitive and non-invasive. The heating unit 33 is disposed on the body 32 for warming up the examinee's finger so as to ensure that the measuring process would not be affected by the lower environment temperature, hence preventing the blood flow to the finger end from stopping. In addition, the heating unit 33 may be disposed on a handheld area of the body 32.

In another embodiment, as shown in FIG. 8, for a better measurement result, the detecting device 3 may be used with another detecting device 3′ and the user may hold the detecting devices 3 and 3′ with both hands. A diversity detecting selecting unit 5 may receive both the output signals SIN1 and SIN2 from the detecting devices 3 and 3′ at the same time, and determine the correct wave shape in the correct signal according to the wave shape of the outputted signals SIN1 and SIN2 to output the vital sign signal SIN_A. As shown in FIG. 9, each of the output signals SIN1 and SIN2 has an incomplete part, and the diversity detecting selecting unit 5 assembles and outputs the rest of the correct parts P1 and P2 as the vital sign signal SIN_A.

The circuit of the analysis device 2 may be integrated in the detecting device 3, and the display unit 34 may display the examining result or perform a tendency prediction according to the examining result. For example, the health condition of the examinee can be shown with flashing of five lights.

In addition, other than the above aspects, the detecting device 3 may also be the current measuring device such as an electrocardiograph (ECG), a SPO2, an electroencephalogram (EEG), an electrogastrogram, or an electromyogram (EMG). The measuring signal outputted by the detecting device 3 includes the frequency band information of a specific organ.

In the above embodiments, the complex electrocardiograph is not needed while analyzing the cardiovascular system. The oxygen concentration signal in blood outputted by a simple oximeter and/or an infrared autonomic nerve scanner as the vital sign signal is good enough to analyze the convincing spectrogram of the rhythm variation of heart rate and entropy graph. Hence the conditions of the organs and the systems (e.g. circulatory system) of the examinee can be determined and evaluated according to the analyzing result.

In addition, after the balance state of various sympathetic nerve and parasympathetic nerve is properly measured, the signal having the measuring result is transmitted to and processed in the ADC unit and the digital processing unit. Thus, the balance states of the autonomic nerve in different systems may be effectively analyzed in real-time.

The detecting device, the analysis device, and the analyzing method of the autonomic nervous state may be used to find out and evaluate the complication or the phenomena caused by the autonomic nerve disorder in different organs or systems of the examinee. Such complication or phenomena are, for example, sudden death caused by the cardiopulmonary disease such as acute myocardia infarction (AMI), neonatal septicemia, depression, central nervous system disease such as epilepsy, and the relaxation after practicing yoga and other treatments such as chigung.

Compared to the above, the traditional electrocardiogram is only a time domain analysis; it cannot provide sufficient information for the diseases that the symptoms show on the electrocardiogram only at an early stage of the disease, such as the premature contraction and premature beat. Moreover, the common biochemical exams can provide a quantitative analysis but these exams need several days to complete.

The detecting device, the analysis device, and the analyzing method of the autonomic nervous state analyze the vital sign of the examinee and conveniently provide a thorough, efficient, easy-to-understand measuring result immediately in real-time. The detecting device, the analysis device, and the analyzing method of the autonomic nervous state may instantly output the quantitative results with precise analyzing value to get to know the body condition of the examinee, who can also self-examine at home.

Additionally, the detecting device, the analysis device, and the analyzing method of the autonomic nervous state may also predict the conditions of the patients with specific occupations such as driver, police officer, military personnel, paramedic, or fireman, to determine whether the body condition is stable for work.

To sum up, the detecting device, the analysis device, and the analyzing method of the autonomic nervous state disclosed in the present invention are to process and analyze the detected vital sign by frequency spectrum analysis or entropy analysis for example, hence the signs of the autonomic nervous state may be further obtained. By the spectrogram or the entropy analyzing result, the organic or systematic states of the human body can be determined.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A detecting device for detecting an autonomic nervous state of an examinee, comprising:
a measuring unit measuring a vital sign of the examinee to output a vital sign signal;
an analog-to-digital converting (ADC) unit coupled to the measuring unit for receiving the vital sign signal, and converting the vital sign from analog to digital to output a digitalized vital sign signal; and
a digital processing unit coupled to the ADC unit for receiving the digitalized vital sign signal and processing the digitalized vital sign signal to obtain a detecting result, the detecting result representing the autonomic nervous state and organic states of the examinee.

2. The detecting device according to claim 1, wherein the digital processing unit performs a frequency calculation and a frequency domain transform on the digitalized vital sign signal to obtain a vital sign time-frequency spectrum as the detecting result, and in the vital sign time-frequency spectrum, a sympathetic nervous state and a parasympathetic nervous state of an autonomic nerve are corresponding to a very low frequency and a low frequency, respectively.

3. The detecting device according to claim 2, wherein the digital processing unit performs a short-time Fourier transform (STFT) or a Hilbert-Huang transform (HHT) on the digitalized vital sign signal to obtain the vital sign time-frequency spectrum.

4. The detecting device according to claim 1, wherein the digital processing unit performs a frequency calculation and an entropy analysis on the digitalized vital sign signal to obtain an entropy analyzing result as the detecting result, and in the entropy analyzing result, a sympathetic nervous state and a parasympathetic nervous state of an autonomic nerve are corresponding to a very low frequency and a low frequency, respectively.

5. The detecting device according to claim 4, wherein after the digital processing unit performs the entropy analysis, a mean square error calculation is carried out to obtain the entropy analyzing result.

6. The detecting device according to claim 1, further comprising:
a recording unit storing a plurality of reference results,
wherein the digital processing unit is coupled to the recording unit for reading the reference results, and compares the detecting result and the reference results to determine whether the autonomic nervous state is abnormal or improved.

7. The detecting device according to claim 1, further comprising:
a body having a concave, wherein the measuring unit is disposed in the concave, and when a finger of the examinee places into the concave and contacts with the measuring unit, the measuring unit measures the pulse of the examinee to output the vital sign signal,
wherein the measuring unit is an infrared measurement device.

8. The detecting device according to claim 1, further comprising:
a heating unit disposed in the body to warm up the finger of the examinee.

9. An analysis device for analyzing an autonomic nervous state used with a measuring device, the measuring device measuring a vital sign of an examinee to output a vital sign signal, the analysis device comprising:
an analog-to-digital converting (ADC) unit receiving the vital sign signal from the measuring device and converting the vital sign signal from analog to digital to output a digitalized vital sign signal; and
a digital processing unit coupled to the ADC unit for receiving the digitalized vital sign signal and processing the digitalized vital sign signal to obtain a detecting result, the detecting result representing the autonomic nervous state and organic states of the examinee.

10. The analysis device according to claim 9, wherein the digital processing unit performs a frequency calculation and a frequency domain transform on the digitalized vital sign signal to obtain a vital sign time-frequency spectrum as the detecting result, wherein in the vital sign time-frequency spectrum, a sympathetic nervous state and a parasympathetic nervous state of an autonomic nerve are corresponding to a very low frequency and a low frequency, respectively.

11. The analysis device according to claim 10, wherein the digital processing unit performs the short-time Fourier transform or the Hilbert-Huang transform on the digitalized vital sign signal to obtain the vital sign time-frequency spectrum.

12. The analysis device according to claim 9, wherein the digital processing unit performs a frequency calculation and an entropy analysis to obtain an entropy analyzing result as the detecting result and in the entropy analysis, a sympathetic nervous state and a parasympathetic nervous state of an autonomic nerve is corresponding to a very low frequency and a low frequency, respectively.

13. The analysis device according to claim 12, wherein after the digital processing unit performs the entropy analysis on the digitalized vital sign signal, a mean square error calculation is carried out to obtain the entropy analyzing result.

14. The analysis device according to claim 9, further comprising:
a recording unit storing a plurality of reference results,
wherein the digital processing unit is coupled to the recording unit for reading the reference results and compares the detecting result and the reference results to determine whether the autonomic nervous state is abnormal or improved.

15. An analyzing method of an autonomic nervous state, comprising:
receiving a vital sign signal from a measuring device, the measuring device measuring a vital sign of an examinee to output the vital sign signal;
converting the vital sign signal from analog to digital to output a digitalized vital sign signal; and
processing the digitalized vital sign signal to obtain a detecting result, the detecting result representing the autonomic nervous state and organic states of the examinee.

16. The analyzing method according to claim 15, wherein the step of processing the digitalized vital sign signal comprising:
performing a frequency calculation and a frequency domain transform to obtain a vital sign time-frequency spectrum as the detecting result, wherein in the vital sign time-frequency spectrum, a sympathetic nervous state and a parasympathetic nervous state of an autonomic nerve is corresponding to a very low frequency and a low frequency, respectively.

17. The analyzing method according to claim 16, wherein the frequency domain transform is a short-time Fourier transform or a Hilbert-Huang transform.

18. The analyzing method according to claim 15, wherein the step of processing the digitalized vital sign signal comprising:
performing a frequency calculation and an entropy analysis on the digitalized vital sign signal to obtain an entropy analyzing result as the detecting result, wherein in the entropy analyzing result, a sympathetic nervous state and a parasympathetic nervous state of an autonomic nerve are corresponding to a very low frequency and a low frequency, respectively.

19. The analyzing method according to claim 18, further comprising:
performing a mean square error calculation to obtain the entropy analyzing result after an entropy analysis is carried out on the digitalized vital sign signal.

20. The analyzing method according to claim 15, further comprising:
reading a plurality of reference results; and
comparing the detecting result and the reference results to determine whether the autonomic nervous state is abnormal or improved.
